(19) United States
(12) Patent Application Publication
(10) Pub. No.: US 2023/0157601 A1
(43) Pub. Date: May 25, 2023

(54) Machine Tool

(71) Applicant: Robert Bosch GmbH, Stuttgart (DE)

(72) Inventors: Joachim Schadow, Stuttgart (DE); Joern Stock, Leinfelden-Echterdingen (DE); Alexander Osswald, Stuttgart (DE); Florian Esenwein, Leinfelden-Echterdingen (DE)

(21) Appl. No.: 16/498,556

(22) PCT Filed: Mar. 23, 2018
(86) PCT No.: PCT/EP2018/057454

§ 371 Date: Jan. 14, 2021

(30) Foreign Application Priority Data
Mar. 30, 2017 (DE) 10 2017 205 430.1

(51) Int. Cl. A61B 5/16; B25F 5/00; A61B 5/18; A61B 5/024

(52) U.S. Cl. CPC A61B 5/165; B25F 5/00; A61B 5/163; A61B 5/18; A61B 5/02405

(57) ABSTRACT

A machine tool, more particularly a hand-held machine tool, includes a drive unit and an open- or closed-loop control unit that is configured to control the drive unit. The drive unit is configured to drive a tool insert that is configured to be accommodated and operated with the machine tool. The machine tool also includes at least one sensor unit with an image sensor. The sensor unit is configured to sense user-specific data and to process the same via the open- or closed-loop control unit such that the drive unit is controlled subject to the sensed data.

The invention relates to a power tool, in particular a hand-held power tool, according to the preamble of the independent claim.

### PRIOR ART

DE 10 2013 202 832 A1 discloses an electrical hand-held power tool having an appliance housing, an appliance-housing coupling means for fastening an ancillary handle to the appliance housing, and a detection means for detecting the ancillary handle on the appliance housing and/or the holding of the ancillary handle, fastened to the appliance housing, by a user, comprising an electrical drive motor, and a control means for controlling the drive motor, having an evaluation means for evaluating the current operating state of the electrical hand-held power tool, and an activation means for activating a protective function if the evaluation means identifies the occurrence of a critical operating situation. The control means is designed to perform the evaluation of the current operating state and/or the activation of the protective function in dependence on the detection of the ancillary handle on the appliance housing and/or the holding of the ancillary handle by the user.

### DISCLOSURE OF THE INVENTION

The invention is based on the object of improving the power tool by simple means.

The object is achieved with a power tool, in particular a hand-held power tool, having a drive unit for driving a receivable insert tool that can be operated by means of the power tool, and having an open-loop or closed-loop control unit for controlling the drive unit by open-loop or closed-loop control.

According to the invention, the power tool has at least one first sensor unit, having a first image sensor, wherein the sensor unit is configured to sense operator-specific data and to process same by means of the open-loop or closed-loop control unit in such a manner that the drive unit is controlled, by open-loop or closed-loop control, in dependence on the sensed data.

The power tool according to the invention renders possible a plurality of advantages that improve the handling of the power tool, in that the operator is observed. In this case, operator-specific data are sensed in order to control the drive unit by open-loop or closed-loop control. It is thus possible, for example, to increase the safety of the operator of the power tool, in that it is monitored, for example, whether safety-relevant accessories are used or are present on the operator and/or on the power tool.

Operator data can be sensed, in particular contactlessly, by means of the sensor unit according to the invention. By means of the image sensor, the operator of the power tool can be observed reliably and, in particular, continuously or at intervals. The data sensed by the image sensor can be evaluated in the form of two-dimensional or three-dimensional images of the environment, or of the immediate surroundings, around the power tool, and the power tool controlled, by open-loop or closed-loop control, in dependence on the evaluated data.

By means of the power tool according to the invention, it can be checked whether the power tool is being operated in a suitable work environment, such that, for example, any light conditions of the work environment can be checked, and the drive unit controlled accordingly by open-loop or closed-loop control.

A “sensor unit” is to be understood to mean an ambient sensor unit, having an ambient sensor element, for sensing at least one environment index quantity that defines an environment surrounding the power tool, defines an effect of the power tool on the surrounding environment and/or defines a positioning of the power tool relative to the surrounding environment. The ambient sensor unit in this case is preferably configured to capture at least one, in particular two-dimensional and/or three-dimensional, image of a surroundings, and to evaluate same, in particular by means of an evaluation unit, for example in the form of a processor. The open-loop or closed-loop control unit in this case may comprise the evaluation unit. The open-loop or closed-loop control unit is preferably configured, at least, to control the drive unit, by open-loop or closed-loop control, in dependence on the at least one drive-unit index quantity sensed by means of a drive-unit sensor unit, and in dependence on the at least one environment index quantity sensed by means of the ambient sensor unit. In addition, the open-loop or closed-loop control unit is preferably designed, at least, to output information to an operator in dependence on the at least one drive-unit index quantity sensed by means of a drive-unit sensor unit, and in dependence on the at least one environment index quantity sensed by means of the ambient sensor unit. Preferably, at least one drive-unit characteristic, a maximum rotational speed, a minimum rotational speed, a maximum torque and/or a minimum torque of the drive unit can by controlled, by open-loop or closed-loop control, by means of the open-loop or closed-loop control unit. For example, in this case the drive unit may be deactivated, or a drive-unit index quantity may be reduced or increased.

The sensor unit may have an objective unit. The objective unit may comprise an optical objective, in particular realized as a converging lens that, in particular, is designed to set and/or alter, in particular to enlarge or reduce, a viewing angle, or image angle, and/or a focal point. The objective unit in this case may allow a viewing angle of at least 120°, in particular at least 180°, preferably at least 270°, more preferably at least 330°, such as, for example, 360°, such that the sensor unit can sense a wide viewing angle range. The objective unit is designed to concentrate and/or scatter light and/or infrared radiation, in order to represent an image of the environment on the image sensor.

An “open-loop or closed-loop control unit” is to be understood to mean, in particular, a unit having a processor unit and having a memory unit, and having an operating program stored in the memory unit. The memory unit may be configured to store the data sensed by the sensor unit. “Configured” is to be understood to mean, in particular, specially programmed, specially designed and/or specially equipped. That an element and/or a unit are/is designed for a particular function, is to be understood to mean, in particular, that the element and/or the unit fulfill/fulfils and/or perform/performs this particular function in at least one application state and/or operating state.

A “drive unit” in this context is to be understood to mean, in particular, a unit designed to generate at least one driving torque and, for the purpose of transmission, to make it available, in particular, to an insert tool. Advantageously, the power tool comprises the drive unit. Particularly advantageously, the drive unit has at least one electric motor. Preferably, the drive unit is configured to drive and/or put into motion at least one insert tool of the power tool.

The power tool is preferably a manually guided, manually operated and/or autonomously operating power tool that is realized as a non-stationary power tool and that is suitable, for example, for DIY-use. In principle, suitable power tools are a non-stationary power tool such as, for example, a hand-held circular saw according to the application DE 3740200 A1, or such as, for example, a knapsack brushcutter according to the application DE 19674764 A1. In addition, garden power tools, in particular lawn mowers, are suitable. Use with hand-held power tools, in particular power drills, power screwdrivers, power grinders or parting-off power tools, is also possible.

An “image sensor” is to be understood to mean a device for capturing an image of an environment index quantity from light and/or infrared radiation that may be effected, in particular, electrically and/or mechanically. The image sensors may have a semiconductor-based structure. The image sensors in this case may sense electromagnetic waves having a wavelength of, for example, 380 to 750 nm (visible light), 780 to 1400 nm (near-infrared), 1400 to 3000 nm (near-infrared) and/or 3000 to 50000 nm (mid-infrared), and expediently evaluate data of the respective wavelength ranges. It is understood that persons skilled in the art know any advantages of sensed data from the respective wavelength ranges, and expediently use these advantages for evaluation of the sensed data.

“Operator-specific” is to be understood to mean relating to the operator, such that, in particular, any sensed data of the operator or of the immediate surroundings of the operator are used to increase the safety of the power tool.

The dependent claims specify expedient developments of the power tool according to the invention.

It may be expedient for the at least one first sensor unit to be configured to sense vital data, in particular a heart rate and/or a fatigue state, of an operator. The sensing of the heart rate makes it possible, for example, to detect whether the operator has a sufficiently high heart rate, and/or to what extent it changes, in particular increases or decreases, during operation of the power tool. If the heart rate is too low, or is even excessive, the drive unit may be switched off, for example, such that the operator temporarily cannot operate the power tool. Moreover, an excessively rapid increase in the heart rate may indicate, for example, that the operator is possibly performing excessively demanding work, such that, for example, the drive unit is controlled accordingly by open-loop or closed-loop control. It is thus ensured that, in the case of prolonged operation of the power tool, the operator has sufficient capacity for the work to be performed.

The at least one first sensor unit may sense the skin of the operator, and determine the heart rate in dependence on a changing skin color. The skin color is more perfused during a heartbeat, and thus usually has a richer color. Between two heartbeats the skin is less perfused, and usually has a paler color. If the first sensor unit senses the skin differences in a specific time interval, the heart rate of the operator can be inferred in dependence on the change in the skin color.

It may additionally be expedient for the at least one first sensor unit to be configured to sense at least one accessory means of the operator, in particular safety eyewear and/or a glove and/or safety footwear. In particular, the at least one first sensor unit may be configured to check whether the operator is wearing the accessory means. It could thus be checked, in a particularly simple manner, whether the operator is using, or wearing, appropriate safety means. Depending on the presence of the at least one accessory means for protecting the operator, the power tool, or the drive unit, could prevent use of the power tool. For example, the power tool may also be put into operation without an accessory means if only a small amount of dust or chips is swirled around in the environment when work is being performed on the workpiece.

Furthermore, it may be expedient for the at least one first sensor unit to be configured to monitor at least one eye, or a lid movement of an eye, of the operator, in order to identify a state of fatigue or injury to the eye. Operator fatigue can thus be identified in a particularly effective manner. It can also be identified whether the operator has possibly sustained an injury to the eye, in that the operator closes the lids of at least one eye, or covers the at least one eye with at least one hand without switching off the power tool.

In particular, the first sensor unit may be configured to sense a swiveling motion, or a turning motion, of the head of the operator. In this case, for example, an eye spacing of both eyes of the operator, or a change in shape of at least one eye contour of an eye, in particular each eye, of the operator may be sensed. This makes it possible to sense, for example, an eye spacing of both eyes of the operator, or a change in shape of at least one eye contour of the operator in such a manner that it is identified whether the viewing direction of the operator is toward the workpiece of the work area on which work is to be performed. The state of attentiveness or the state of fatigue of the operator during operation of the power tool can thus be inferred. Besides identification of fatigue, further aspects of the operator and/or of the work surroundings may also be analyzed, such as, for example, a pupil width of at least one eye of the operator, in particular in combination with the ambient brightness of the work area. For example, deficient light conditions of the work areas may be inferred if the pupil size, in the case of deficient light conditions, is comparatively large in comparison with the pupil size in the case of good light conditions.

Alternatively or additionally, the first sensor unit may be configured to sense a swiveling motion of at least one pupil of an eye, in particular each eye, of the operator. In this case, for example, a pupil spacing of both pupils of the operator, or a change in shape of at least one pupil contour, in particular each pupil contour, of the operator may be sensed in such a manner that it is identified whether the viewing direction of the operator is toward the workpiece of the work area on which work is to be performed. The first sensor unit may be arranged and oriented on the power tool in such a manner that the pupil contour of an eye of the operator that is directed toward the workpiece on which work is to be performed is, for example, substantially circular, and the pupil contour of an eye of the operator that is directed away from the workpiece on which work is to be performed is, for example, substantially elliptical. In principle, however, there are also other possible measuring methods by which a viewing direction of the operator can be sensed by means of a sensor unit having an image sensor.

For the purpose of sensing corresponding operator-specific data such as, for example, a change in shape of a pupil, the sensor unit many be arranged in a fixed manner on the power tool, in such a manner that a change in the operator-specific data may be inferred in dependence on a relative movement of operator-specific data such as, for example, the pupil size, with respect to the sensor unit.

“Elliptical” is to be understood to mean a geometric shape of an ellipse, having a main axis that defines a large diameter of the ellipse, and having a secondary axis, which is at right angles to the main axis and which defines a small diameter of the ellipse. The main axis defines the maximum extent of the ellipse. The secondary axis defines the minimum extent of the ellipse. “Substantially circular” is to be understood to mean a geometric shape of a circle having a constant diameter, that, with respect to an elliptical shape, in particular has a maximum deviation of maximally 10%, in particular maximally 5%, preferably maximally 2%, and accordingly a main axis of the circle has a maximum longitudinal deviation of 10%, in particular of 5%, preferably of 2%, with respect to a secondary axis of the circle.

An “eye contour” is to be understood to mean, in particular, a contour that defines the eye of the operator and in particular is delimited, for example, by the eyelids. A pupil contour is to be understood to mean, in particular, a contour that defines the pupil of the eye of the operator and in particular is delimited, for example, by the iris of the eye.

Furthermore, it may be expedient for the at least one first sensor unit to be configured to sense a face of the operator, and in particular to identify whether the operator is authorized to operate. In this case, for example, an operator's face, or individual, in particular distinctive parts of an operator's face, may be sensed by means of the sensor unit, and stored in a memory unit. Before and/or upon operation of the power tool, the sensor unit may sense the face or individual parts of the face of the operator and, by means of the stored data, effect a comparison in order to enable or inhibit operation of the power tool.

It is thus particularly easy to define an operator circle of operators authorized to actuate, or operate, the power tool. It is thus possible to achieve both reliable theft prevention and reliable child safety, since the operators that do not belong to the operator circle are excluded from operation of the power tool.

It is proposed that the at least one first sensor unit be configured to observe a work area, in order to control the drive unit, by open-loop or closed-loop control, in dependence on a body part of an operator present in the work area, or a person present in the work area. It can thus be determined particularly easily whether a part of an operator, or another person, is present in the work area of the power tool.

It is additionally proposed that the at least one first sensor unit be configured to sense an actual state of the power tool, in at least one work step, before and/or while the drive unit is in the operating state, and to compare it with a reference state, and to control the drive unit, by open-loop or closed-loop control, in dependence on a deviation by the actual state in comparison with the reference state. A “work step” is to be understood to mean a step in the performing of work on a workpiece, in which the drive unit is switched on and is operated. Before the work step, is to be understood to mean that the drive unit is not yet in an operating state, but the operator is already holding the power tool in an operating position or is gripping the power tool by at least one hand, or is already actuating a switch of the power tool in such a manner that the drive unit is not being put into the operating state.

It is further proposed that the at least one first sensor unit be configured to identify whether the operator actuates the power tool. “Actuates” is to be understood to mean that the operator puts the power tool into an operating state, and preferably holds the power tool, at least by one hand. In this case, for example, the viewing angle of the image sensor may be designed in such a manner that it is sensed whether a hand of the operator has been placed on, or is gripping, a grip region of the power tool. It can thus be identified, in a particularly simple manner, whether the operator is yet holding or gripping the hand-held power tool. It is consequently possible to provide a further or additional device that, for example in addition to the existing devices for identifying an inadmissible operating state such as, for example, in the case of inadvertent dropping of the power tool, deactivates the drive unit. A restart lockout can thus be achieved in a reliable manner. In particular, in the case of a dropped power tool, the drive unit may be deactivated even while the power tool is falling.

It may be expedient for the power tool to have at least one second sensor unit, which is spaced apart from the first sensor unit, wherein the first sensor unit is oriented toward an operator of the power tool, and the second sensor unit is oriented toward the work area. In particular, the at least one second sensor unit may be configured to observe a work area, in order to control the drive unit, by open-loop or closed-loop control, in dependence on a body part of an operator present in the work area, or on a person present in the work area. Thus, both the operator and the work area can be sensed. It is thus possible, for example, to identify whether the work area is inadmissible for performance of work, in that it is sensed, for example, whether the work area is adequately illuminated and/or whether the operator is looking at the work area while the power tool is in operation.

Observation of the work area makes it possible to prevent incorrect operation of the power tool, for example in that it is identified that a drilling tool is not correctly clamped-in, or that a hand of the operator or of another person comes too close to an operating saw blade.

In particular, in the case of a measuring device such as a laser distance meter, the laser beam may be deactivated as soon as a face of a person is sensed in the target area of the laser. Preferably, in the case of a lawn mower, obstacles such as, for example, persons, in particular children, or animals, in particular domestic pets, may be sensed and rapidly and unambiguously identified, in order to deactivate the power tool or bypass the obstacles.

It may additionally be expedient for the first and/or the second sensor unit to sense data in the infrared radiation range. Hazard sources such as, for example, people or animals can thus be identified particularly easily. In particular, an accessory device such as, for example, protective eyewear or a glove may have a reflector such as, for example, a retro-reflector, that in particular is configured to reflect infrared radiation in, for example, a wavelength range of from 780 nm to 1400 nm, and/or 1400 nm to 3000 nm, and/or from 3000 nm to 50000 nm. Identification of the accessory device can thus be facilitated by means of the first or the second sensor unit. The reflector in this case is intended to reflect, or radiate back, incident radiation of a defined wavelength such as, for example, a wavelength in the infrared radiation range. The sensor unit may accordingly pick up the reflected radiation, and this may then be evaluated by an evaluation unit.

Preferably, the power tool and/or the first sensor unit may have an infrared radiation means that is designed to emit infrared radiation. In particular, the infrared radiation to be emitted by the infrared radiation means may be reflected on the reflector. Preferably, the radiation reflected by the reflector may be sensed by means of the image sensor of the first or the second sensor unit.

Furthermore, it may be expedient for the power tool to have at least one communication unit for communicating with at least one external unit, for the purpose of exchanging electronic data, at least for the purpose of controlling the drive unit by open-loop or closed-loop control. The communication unit is preferably realized as a wireless communication unit. The communication unit in this case may be realized as a WLAN communication unit, as a Bluetooth communication unit, as a radio communication unit, as an RFID communication unit, as an NFC unit, as an infrared communication unit, as a mobile radio telephony network communication unit, or the like. Particularly, preferably, the open-loop or closed-loop control unit is designed to control the drive unit and/or safety functions, by open-loop or closed-loop control, in dependence on the at least one environment index quantity sensed by means of the ambient sensor unit, and in dependence on electronic data transmitted to the open-loop or closed-loop control unit by means of the communication unit. Particularly preferably, the communication unit is designed for bidirectional data transmission. In an alternative design, the communication unit is realized as a wired communication unit, such as, for example, as a LAN communication unit, as a USB communication unit, or the like. The external unit is preferably realized as a smartphone, which has an app for communicating with the communication unit. It is also conceivable, however, for the external unit to be realized as an external, transportable operating unit, as a fixedly installed operating unit at an operator's workplace, as a synchronization unit of a deployment location that is fixedly installed in a room and that can be controlled by a control centre, such as, for example, on the basis of company specifications/safety requirements, as a unit for monitoring index quantities of the body of an operator, as an external sensor unit, or as another operating unit, input station and/or centralized or decentralized terminal. Thus, advantageously, synchronization of electronic data is made possible. If, for example, the power tool is put into operation in a synchronization mode, for example by insertion of a rechargeable battery device, upon insertion of an electric power supply cable or by activation by an operator, a connection is established, at least partly automatically, between the communication unit and the external unit. Settings stored in the external unit can thus be transmitted, preferably directly, to the power tool. The settings in this case may be individual settings of an operator, such as, for example, a desired rapid acceleration to a set rotational speed and a maximum power, and/or company specifications such as, for example, compliance with a safety function in a defined area of a company premises or deployment location, etc.

Moreover, electronic data may be transmitted to the external unit by means of the communication unit. In this case, for example, there may be transmitted to a company control centre, or the like, an operator's exposure to vibration, for the purpose of monitoring compliance with an exposure limit, and/or any payment of allowances and/or a run-time and a load, for the purpose of assessing utilization of a power tool. Moreover, it is conceivable for the external unit to verify the presence of an item of safety equipment and/or suitable work clothing, such as, for example, by means of RFID identification, etc., the external unit transmitting settings to the open-loop or closed-loop control unit, via the communication unit, in dependence on an identified item of safety equipment and/or suitable work clothing, for the purpose of controlling the drive unit by open-loop or closed-loop control.

Furthermore, it may be expedient for the power tool to have at least one information output unit, which is configured to provide information to the operator of the power tool in dependence on the sensed data, in particular by means of an optical, haptic and/or acoustic signal. An information output unit for outputting information to an operator is preferably realized as an optical, acoustic and/or haptic information output unit. The information output unit in this case is preferably a constituent part of the power tool. It is also conceivable, however, for the information output unit to be a constituent part of an external unit such as, for example, a smartphone, tablet, PC, laptop or the like. For the purpose of outputting information to an operator, the information output unit preferably comprises at least one optical output unit such as, for example, an LC display, a touch-sensitive display, an LED display, a plasma display or the like, for optical output of information to an operator. Preferably, the information output unit comprises at least one acoustic output unit such as, for example, a loudspeaker or the like, for acoustic output of information to an operator. Particularly preferably, the information output unit comprises at least one haptic output unit such as, for example, a vibration generator unit or the like, for haptic output of information to an operator. It is also conceivable, however, for output of information to an operator to be effected as a result of the drive unit being operated by means of the open-loop and/or closed-loop control unit. It is conceivable in this case for output of information to an operator to be effected, for example, as a result of a fluctuation of a drive-unit rotational speed. Further drive-unit related outputs of information to an operator that are considered appropriate by persons skilled in the art are likewise conceivable.

The invention also relates to a garden power tool, in particular a lawn mower.

The invention additionally relates to a hand-held power tool, in particular a power drill, a power screwdriver, a power grinder or a parting-off tool.

The invention also relates to a system having at least one power tool that has at least one accessory means that can be sensed by the sensor unit.

### BRIEF DESCRIPTION OF THE DRAWINGS

Further advantages are evident from the following description of the drawing. The drawing shows exemplary embodiments of the invention. The drawings, the description and the claims contain numerous features in combinations. Persons skilled in the art will expediently also consider the features individually, and combine them to form appropriate further combinations. There are shown:

FIG. 1 a schematic representation of a first embodiment of a power tool according to the invention,

FIG. 2 a schematic representation of a second embodiment of a power tool according to the invention, and

FIG. 3 a schematic representation of a third embodiment of a power tool according to the invention.

In the following figures, components that are the same are denoted by the same reference numerals.

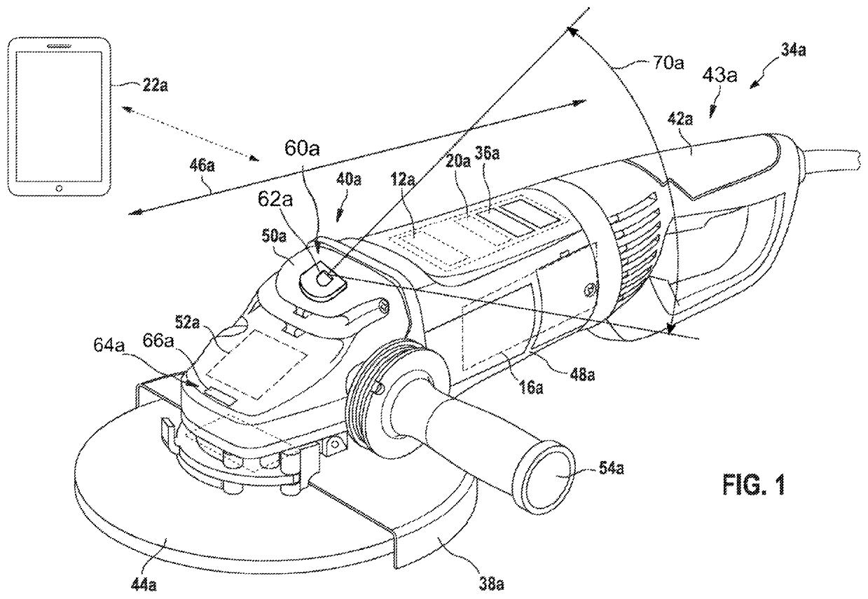

FIG. 1 shows a portable power tool 34a. In this case the power tool 34a is realized as an angle grinder. The power tool 34a comprises at least one accessory device 38a, realized as a protective hood. The power tool 34a additionally comprises at least one power-tool housing 40a, and a main handle 42a, which extends, in the direction of a direction of main extent 46a of the power tool 34a, on a side of the power-tool housing 40a that faces away from an insert tool 44a. The insert tool 44a in this case is realized as a grinding disk. It is also conceivable, however, for the insert tool 44a to be realized as a parting-off or polishing disk. The power-tool housing 40a comprises a motor housing 48a for accommodating a drive unit 16a of the power tool 34a. The power-tool housing 40a additionally comprises a transmission housing 50a for accommodating an output unit 52a of the power tool 34a. The drive unit 16a is designed to drive the insert tool 44a in rotation, via the output unit 52a. Arranged on the transmission housing 50a is a further accessory device 54a, realized as an ancillary handle unit. The accessory device 54a realized as an ancillary handle unit extends transversely in relation to the direction of main extent 46a of the power tool 34a.

The power tool 34a preferably comprises a mains-operated energy supply device. Alternatively or additionally, the power tool 34a can be operated, independently of mains power operation, by means of a rechargeable battery pack.

The power tool 34a additionally comprises at least one open-loop or closed-loop control unit 12a for controlling the drive unit 16a by open-loop or closed-loop control.

The power tool 34a has at least one first sensor unit 60a, having a first image sensor 62a, which is configured to sense operator-specific data of the operator and to evaluate same by means of an evaluation unit (not shown). The drive unit 16a is controlled, by open-loop or closed-loop control 12a, in dependence on the sensed and evaluated data.

The first image sensor 62a is configured to sense vital data, in particular a heart rate and/or a fatigue state, of an operator. It may be sensed in this case, for example, whether the operator has a sufficiently high heart rate, and/or to what extent the heart rate changes, in particular increases or reduces, during operation of the power tool.

The first image sensor 62a is configured to sense at least one accessory means of the operator, realized as protective eyewear and/or as a glove and/or as protective clothing. Alternatively or additionally, the first image sensor 62a may be configured to sense an accessory means realized as safety footwear. By means of the first image sensor 62a, it can be checked whether the operator is wearing the accessory means.

The first sensor unit 60a and/or the first image sensor 62a in this case are/is arranged on a side of the power-tool housing 40a that faces away from the insert tool 44a so as to sense the accessory means of the operator, in at least one work step, before and/or while the drive unit 16a is in the operating state.

The first image sensor 62a is configured to monitor at least one eye or a lid movement of an eye of the operator, in order to identify a fatigue state or an injury to the eye.

The first image sensor 62a is configured to sense a swiveling motion, or a turning motion, of the head of the operator. For example, an eye spacing of both eyes of the operator, or a change in shape of at least one eye contour of an eye, in particular each eye, of the operator may be sensed. This makes it possible to evaluate, by means of an evaluation unit, whether a viewing angle of the operator is directed toward the work area.

The first image sensor 62a is configured to sense a face of the operator, and to identify whether the operator is authorized to operate. In this case, a face, or individual distinctive face parts of the operator, is sensed by means of the first image sensor 62a and stored in a memory unit (not represented). Upon operation of the power tool, the first image sensor 62a may sense the face, or individual face parts of the operator, and effect a comparison by means of the stored data, in order to enable operation of the power tool 34a if the operator is authorized to operate, or to inhibit operation in the case of an operator not authorized to operate.

The image sensor 62a is additionally configured to observe a work area, in order to control the drive unit 12a, by open-loop or closed-loop control, in dependence on a body part of an operator present in the work region, or a person present in the work region. The image sensor 62a in this case may be arranged on the power tool 34a in such a manner that, for example, a 360° viewing angle is obtained, preferably with use of an appropriate objective unit.

The first image sensor 62a is configured to sense an actual state of the power tool 34a, in at least one work step, before and while the drive unit 16a is in the operating state, and to compare it with a reference state, and to control the drive unit 16a, by open-loop or closed-loop control, in dependence on a deviation by the actual state in comparison with the reference state.

In this case, the first image sensor 62a senses operator-specific data, in at least one work step, before and/or while the drive unit 16a is in the operating state.

The first image sensor 62a is configured to identify whether the operator is actuating the power tool 34a, in that at least one range of a viewing angle 70a of the first image sensor 62a is directed toward the grip region 43a of the main handle 42a of the power tool. It can thus be sensed whether a hand of the operator is gripping the grip region 43a.

The power tool 34a has a second sensor unit 64a, having a second image sensor 66a. The second image sensor 66a is spaced apart from the first image sensor 62a. The first image sensor 62a is oriented substantially toward the operator of the power tool 34a. The second image sensor 66a is oriented toward the work area of the power tool 34a. A second image sensor 66a that is directed toward the work region makes it possible to identify what insert tool 44a is being used and/or whether, for example, the accessory means 38a of the power tool 34a, realized as a protective hood, is being used.

The first image sensor 62a preferably senses data in the infrared radiation range, such that hazard sources such as, for example, people or animals, can be identified in a simple and reliable manner.

The power tool 34a has a communication unit 20a for communicating with an external unit 22a, for the purpose of exchanging electronic data, for the purpose of controlling the drive unit 16a by open-loop or closed-loop control. The communication unit 20a is preferably realized as a wireless communication unit 20a. In addition, electronic data can be transmitted to the external unit 22a by means of the communication unit 20a.

The external unit 22a is preferably realized as a smartphone, which has an app for communicating with the communication unit 20a.

The power tool 34a has at least one information output unit 36a, which is configured to provide information to the operator of the power tool 34a in dependence on the sensed data, in particular by means of an optical, haptic and/or acoustic signal.

In addition, the open-loop and/or closed-loop control unit 12a outputs at least one item of information, by means of the information output unit 36a of the power tool 34a, in dependence on data sensed by a power-tool sensor, and/or data communicated by means of a communication unit, in particular for the purpose of providing information to the operator concerning a state of the power tool and/or warning against a hazard. Furthermore, the open-loop or closed-loop control unit 12a controls at least one operating-mode setting of the power tool 34a, by open-loop or closed-loop control, in dependence on data communicated by means of a communication unit.

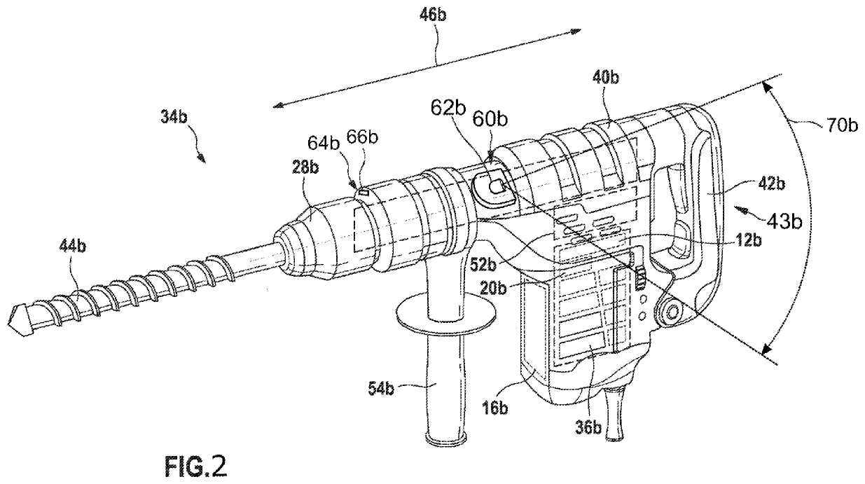

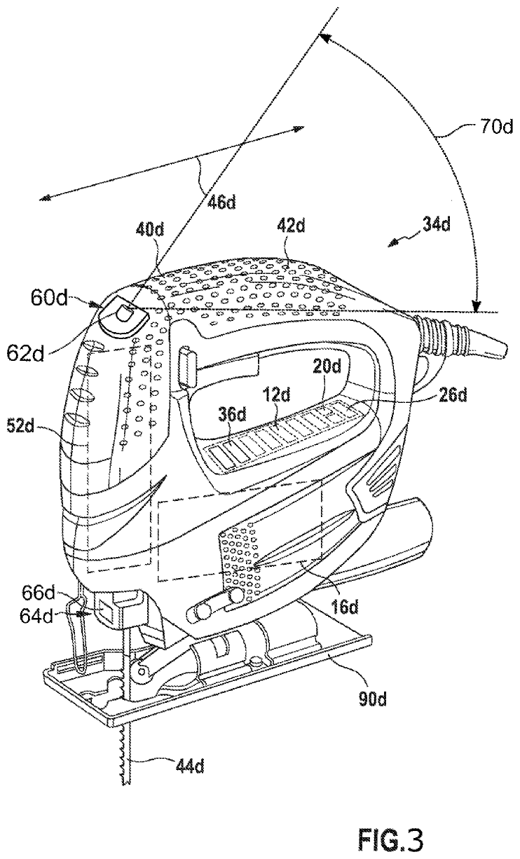

FIGS. 2 to 3 show further exemplary embodiments of the invention. The description and the drawing that follow are limited substantially to the differences between the exemplary embodiments, and in principle reference may also be made to the drawings and/or the description of the other exemplary embodiments, in particular of FIG. 1, in respect of components that have the same designation, in particular in respect of components denoted by the same references. To distinguish the exemplary embodiments, the letter a has been appended to the references of the exemplary embodiment in FIG. 1. In the exemplary embodiments of FIGS. 2 to 3, the letter a has been replaced by the letters b or d.

FIG. 2 shows a power tool 34b realized as a hammer drill and/or chiseling hammer. The power tool 34b comprises at least one percussive mechanism device (not shown). The power tool 34b additionally comprises a power-tool housing 40b, arranged on which, in a front region, there is a tool receiver 28b of the power tool 34b for receiving an insert tool 44b. On a side that faces away from the front region, the power tool 34b comprises a main handle 42b for guiding the power tool 34b and for transmitting a force, in particular a pressure force, from an operator to the power tool 34b. The power tool 34b is additionally realized with a detachable ancillary handle unit. The ancillary handle unit in this case may be fastened to the power-tool housing 40b by means of a latching connection, or other connections considered appropriate by persons skilled in the art.

The first sensor unit 60b and/or the first image sensor 62b are arranged on a lateral surface of the power-tool housing 40b, and are arranged, at least partly, on a side of the power-tool housing 40b that faces away from the insert tool 44b, for the purpose of sensing the accessory means of the operator, in at least one work state, before and/or while the drive unit 16b is in the operating state.

The power tool 34b has a second sensor unit 64b, having a second image sensor 66b. The second image sensor 66b is spaced apart from the first image sensor 62b. The first image sensor 62b is oriented substantially toward the operator of the power tool 34b. The second image sensor 66b is oriented toward the work area of the power tool 34b. A second image sensor 66b that is directed toward the work area makes it possible to identify what insert tool 44b is being used and/or whether, for example, the accessory means 38b of the power tool 34b, realized as a protective hood, is being used.

FIG. 3 shows a power tool 34d realized as a jigsaw. The power tool 34d has a power-tool housing 40d, which surrounds a drive unit 16d of the power tool 34d and an output unit 52d of the power tool 34d. The drive unit 16d and the output unit 52d are designed to drive an insert tool 44d, clamped in a tool receiver (not shown) of the power tool 34d, in an oscillating manner. In this case, the insert tool 44d is driven in an oscillating manner substantially perpendicularly in relation to a working direction. The insert tool 44d is realized as a jigsaw blade. Oscillating driving of the insert tool 44d in this case is effected in a manner already known to persons skilled in the art.

The first sensor unit 60d and/or the first image sensor 62d are/is arranged on a side of the power-tool housing 40d that faces away from the insert tool 44d, so as to sense the accessory means of the operator, in at least one work step, before and/or while the drive unit 16d is in the operating state.

The power tool 34d has a second sensor unit 64d, having a second image sensor 66d. The second image sensor 66d is spaced apart from the first image sensor 62d. The first image sensor 62d is oriented substantially toward the operator of the power tool 34d. The second image sensor 66d is oriented toward the work area of the power tool 34d. A second image sensor 66d that is directed toward the work area makes it possible to identify what insert tool 44d is being used and/or whether, for example, the accessory means 38d of the power tool 34d, realized as a protective hood, is being used.

1. A power tool, comprising:
a drive unit configured to drive a receivable insert tool that is configured to be operated by the power tool;
an open-loop or closed-loop control unit configured to control the drive unit by open-loop or closed-loop control; and
at least one first sensor unit, having a first image sensor, the at least one first sensor unit configured to sense data that is specific to an operator and to process the sensed data via the open-loop or closed-loop control unit in such a manner that the drive unit is controlled, by open-loop or closed-loop control, in dependence on the sensed data.

2. The power tool as claimed in claim 1, wherein the at least one first sensor unit is configured to sense vital data of the operator.

3. The power tool as claimed in claim 1, wherein the at least one first sensor unit is configured to sense at least one accessory of the operator.

4. The power tool as claimed in claim 1, wherein the at least one first sensor unit is configured to monitor at least one eye, or a lid movement of an eye, of the operator in order to identify a state of fatigue or injury to the eye.

5. The power tool as claimed in claim 1, wherein the at least one first sensor unit is configured to sense a face of the operator.

6. The power tool as claimed in claim 1, wherein the at least one first sensor unit is configured to observe a work area in order to control the drive unit, by open-loop or closed-loop control, in dependence on a body part of the operator present in the work area or a person present in the work area.

7. The power tool as claimed in claim 1, wherein the at least one first sensor unit is configured to:
sense an actual state of the power tool, in at least one work step, one or more of before the drive unit is in the operating state and/or while the drive unit is in the operating state,
compare the actual state with a reference state, and
control the drive unit, by open-loop or closed-loop control, in dependence on a deviation by the actual state in comparison with the reference state.

8. The power tool as claimed in claim 7, wherein the at least one first sensor unit is configured to identify whether the operator actuates the power tool.

9. The power tool as claimed in claim 1, further comprising at least one second sensor unit, that is spaced apart from the first sensor unit, wherein the first sensor unit is oriented toward the operator of the power tool, and the second sensor unit is oriented toward a work area.

10. The power tool as claimed in claim 9, wherein one or more of the first sensor unit and the second sensor unit senses data in the infrared radiation range.

11. The power tool as claimed in claim 1, further comprising at least one communication unit for configured to communicate with at least one external unit, so as to exchange electronic data, at least to control the drive unit by open-loop or closed-loop control.

12. The power tool as claimed in claim 1, further comprising at least one information output unit, that is configured to provide information to the operator of the power tool in dependence on the sensed data.

13. The power tool as claimed in claim 1, wherein the power tool is configured as a hand-held power tool.

14. The power tool as claimed in claim 2, wherein the vital data is one or more of a heart rate and a fatigue state.

15. The power tool as claimed in claim 3, wherein the at least one accessory of the operator is one or more of safety eyewear, a glove, and safety footwear.

16. The power tool as claimed in claim 15, wherein the at least one first sensor unit is configured to check whether the operator is wearing the at least one accessory.

17. The power tool as claimed in claim 5, wherein the at least one first sensor unit is configured to identify whether the operator is authorized to operate.

18. The power tool as claimed in claim 12, wherein the at least one information output unit is configured to provide the information to the operator of the power tool in dependence on the sensed data by one or more of an optical signal, a haptic signal, and an acoustic signal.
